(19) United States
(12) Patent Application Publication
(10) Pub. No.: US 2013/0203009 A1
(43) Pub. Date: Aug. 8, 2013

(54) DENTAL REGISTRATION DEVICE AND METHOD

(71) Applicants: Amnon Mutsafi, Sitria (IL); Ronny Winshtein, Ramat Hasharon (IL)

(72) Inventors: Amnon Mutsafi, Sitria (IL); Ronny Winshtein, Ramat Hasharon (IL)

(21) Appl. No.: 13/824,683

(22) PCT Filed: Oct. 24, 2011
(86) PCT No.: PCT/IL11/00821

§ 371 Date: Mar. 18, 2013

Related U.S. Application Data
(60) Provisional application No. 61/407,166, filed on Oct. 27, 2010.

(51) Int. Cl. A61C 19/05; A61C 9/00; A61B 6/12; A61B 5/00; A61C 8/00; A61B 6/14

(52) U.S. Cl. CPC A61C 19/05; A61C 8/0001; A61B 6/14; A61B 6/12; A61B 5/0088; A61C 9/0006; A61C 8/0033
USPC 433/27; 433/68; 433/71; 433/29; 433/36

(57) ABSTRACT

An apparatus for registering occlusal dimensions, comprising a support surface sized and configured for providing areal support to a registration rim; and at least one connector adapted to releasably connect to a dental implant extension.

### RELATED APPLICATION/S

This application claims the benefit of priority under 35 USC 119(e) of a U.S. provisional application Ser. No. 61/407,166, filed Oct. 27, 2010 and having title “BITE REGISTRATION DEVICE AND METHOD”.

The contents of the above document are incorporated by reference as if fully set forth herein.

### FIELD OF THE INVENTION

The present invention generally relates to systems and methods for facilitating and/or assisting in oral or dental measurements, and more specifically to methods and devices for recording occlusal dimensions.

### BACKGROUND OF THE INVENTION

Teeth replacement is a vital procedure intended for treating or preventing oral conditions such as volumetric change of soft and hard tissues, shifting of surrounding teeth, damage to roots of adjacent teeth and deterioration of jawbone, as well as aesthetic issues. Dental practice includes numerous implants, devices and methods for replacing teeth. Two most common practices are bite registration and dental impression, where in both cases an elastic, usually crosslinking, material is used to copy part or all of a person's dentition (hard as well as soft tissue) and other areas of the mouth. Much time is invested both by dentists and dental technicians for preparing accurate crown implants, due to the complexity involved. Registration and impression procedures, especially in multiple teeth replacements, usually end up with some inaccuracies and much time is invested usually due to several trial-and-error occurrences. Such inaccuracies may cause misalignment of bite and jaws, resulting in improper functionality, aesthetics and possibly even temporomandibular joint disorder (TMJ), a painful condition that can affect head, neck, jaw, and shoulders.

Therefore there is still a need for improved devices, instrumentation and methods which may produce more accurate and fast dentition copying for teeth replacement procedures using dental implants, especially in multiple teeth replacements.

The main principle behind the current invention suggests the use of accurate bite registration devices that are selectively adjusted in at least one axis (e.g., height), the fixation connection between the transfers and lip support device, and may preferably be used also as impression copings. In multiple teeth replacement procedures, a plurality of such devices may be used in order to preserve proper jaw occlusion, teeth alignment and lip support. Accordingly, a multiple-point bite registration in a fixed coordinate system is suggested as an important tool for to the dental impression procedure.

### SUMMARY OF THE INVENTION

In a broad aspect of some embodiments there is provided an apparatus for registering occlusal dimensions. In some embodiments, the apparatus includes a support surface sized and configured for providing areal support to a registration rim; and at least one connector adapted to releasably connect to a dental implant extension. In some embodiments of the invention, the apparatus includes a registration base member having the support surface at a first end and the at least one connector at a second end opposing the first end.

The registration base may include an occlusal flat solid connectable against a free end of the dental implant extension thereby providing the support surface in occlusal position. In some embodiments of the invention, the occlusal flat solid is sized and configured for positioning over a chosen bare gingival length, optionally in-between two laterally positioned teeth. Optionally, the occlusal flat solid and/or the registration rim is sized for registering a single opposing tooth.

In some embodiments of the invention, the registration base member includes a vestibular flat solid laterally connectable to the dental implant extension thereby providing the support surface in vestibular position. Optionally, the vestibular flat solid is textured and configured to harmlessly press against supported soft tissue, for example labial mucosa and/or buccal mucosa. In some embodiments of the invention, the registration base member includes a mesh. Optionally the registration base member includes an inscribable and/or cuttable portion.

In some embodiments of the invention, the registration rim includes impression material, which may contain at least one of wax, acrylic material, composite resin, alginate, silicone, polyether, and hydrocolloid. In some embodiments, the registration rim includes a deformable imprinting block. Alternatively or additionally, the registration rim includes at least one pressure sensor. Optionally, the registration base member and/or registration rim includes radiopaque portion and/or marker and/or an identification marking and/or light absorbing and/or dissipating portion. In some embodiments of the invention, the impression material readily fills a rigid container having an opening, which may or may not be provided covered with a removable covering. In some embodiments of the invention, the registration rim includes thickness less than is 10 mm or less, optionally 7 mm or less (for example, an average and/or a maximal thickness of 1 mm or 2 mm).

In some embodiments of the invention, the dental implant extension includes an impression coping or a transfer. Optionally, the dental implant extension is length and/or angularly adjustable. In some embodiments of the invention, the dental implant extension includes a housing member having lumen and a moving member selectively positionable along the lumen. Optionally, the dental implant extension includes means to level the support surface. Optionally, the dental implant extension includes locking means for fixating a chosen geometry. In some embodiments of the invention, the dental implant extension includes at least one docking arrangement for securing the connector in place. The docking arrangement may be positionable along a length of the dental implant extension and optionally include at least one recess.

In some embodiments of the invention, the at least one connector includes a snap connection. Optionally, the connector is length adjustable. In some embodiments of the invention, the connector is movable along a track provided on the second end of the registration base member.

In some embodiments of the invention, the registration base member is bendable, optionally malleable to a chosen contour. The registration base member may be elastically deformable or plastically deformable. In some embodiments, the registration base member includes a marking (for example, flattening, coloring or any symbol) at its vestibular side for directing assembly direction.

In a broad aspect of some embodiments, there is provided a modular system for registering occlusal dimensions. In some embodiments, the modular system includes a plurality of dental implant extensions; at least one occlusal flat solid connectable to at least one of the dental implant extensions; and/or at least one vestibular flat solid connectable to at least two of the dental implant extensions.

In some embodiments of the invention, the modular system includes a fixating member connectable to at least one of the dental implant extension and adapted for fixating chosen tridimensional geometric relations between at least two dental implant extensions connected thereto. In some embodiments of the invention, the fixating member is sized and shaped for lingual and/or palatal placement or alternative or additionally for occlusal and/or buccal placement. Optionally, the fixating member is firmly connectable to the at least one dental implant extension at or in-between two points along a length thereof.

In a broad aspect of some embodiments there is also provided a method of registering at least one occlusal dimension. In some embodiments of the invention, the method includes at least one of the following steps (not necessarily in same order): fastening a plurality of dental implant extensions to a corresponding number of dental implants; connecting at least one occlusal flat solid includes an occlusal support surface to at least one of the dental implant extensions; and adjusting height of the occlusal support surface according to a chosen occlusion.

Optionally, the height adjusting includes replacing the at least one dental implant extension with another dental implant extension having a chosen length and/or changing a length of the at least one dental implant extension. In some embodiments, another optional step may include providing (optionally, locking in position) an impression rim on the occlusal support surface. In some is embodiments of the invention, another optional step may include fixating the plurality of dental implant extensions in chosen tridimensional geometric relations using a fixating member. In some embodiments, the fixating member is placed in a lingual and/or palatal position, or additionally or alternatively, in occlusal or buccal position. Another optional step may include laterally connecting a malleable vestibular flat solid to at least two of the dental implant extensions. In some embodiments of the invention, the vestibular flat solid includes length adjustable connectors, and the method may further include selectively modifying length to at least one of the length adjustable connectors.

In a broad aspect of some embodiments there is also provided a method for imprinting a jaw, optionally following at least one step of the method for registering the at least one occlusal dimension. In some embodiments, the method includes at least one of the following steps (not necessarily in same order): pressing an impression tray filled with impression material against the plurality of dental implant extensions; and withdrawing the impression tray. In some embodiments of the invention, at least one occlusal flat solid is removed or alternatively kept in place.

In a broad aspect of some embodiments there is also provided a method for imprinting a jaw, optionally following at least one step of the method for registering the at least one occlusal dimension. In some embodiments, the method includes at least one of the following steps (not necessarily in same order): pressurizing settable impression material to covering the plurality of dental implant extensions along a chosen bare gingival length; allowing the settable impression material to harden; and removing the hardened impression material. In some embodiments of the invention, the method includes the step of unfastening the plurality of the dental implant extensions from the corresponding number of dental implants.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B illustrate schematic isometric views of a registration base member before and after registration rim deploying, in accordance with embodiments of the invention;

FIG. 2 illustrates a schematic exploded view of an exemplary modular system for registering occlusal dimensions, in accordance with embodiments of the invention;

FIG. 3 illustrates a schematic cutaway view of an exemplary occlusal registration device, in accordance with embodiments of the invention;

FIGS. 4A-C illustrate different schematic cutaway views of exemplary transfers and deployments thereof, in accordance with embodiments of the invention;

FIG. 5 schematically illustrates a cutaway view of an exemplary adjustable occlusal registration device, in accordance with embodiments of the invention;

FIGS. 6A-D schematically illustrate an exemplary vestibular flat solid and deployments thereof, in accordance with embodiments of the invention;

FIGS. 7A-D schematically illustrate several steps of a method of dental impression using an exemplary occlusal registration device during multiple teeth replacement procedure, in accordance with embodiments of the invention;

FIG. 8 schematically illustrates front view of exemplary occlusal stamping and registering devices deployed for impression two missing opposing teeth, in accordance with embodiments of the invention;

FIG. 9 schematically illustrates exemplary interconnected occlusal registering devices deployed for impression an entire teeth line, in accordance with embodiments of the invention;

FIGS. 10A-B schematically illustrate views of different exemplary connections elements between a registration base member and an impression rim, in accordance with embodiments of the invention; and

FIGS. 11A-E illustrate different views of an exemplary modular system incorporating at least one single-tooth occlusal registration device, and members thereof, in accordance with embodiments of the invention.

### DETAILED DESCRIPTIONS OF EXEMPLARY EMBODIMENTS

The following preferred embodiments may be described in the context of exemplary dental occlusal registration and/or impression procedures for ease of description and understanding. However, the invention is not limited to the specifically described devices and methods, and may be adapted to various clinical applications without departing from the overall scope of the invention.

It is to be understood that the terminology used herein is used for the purpose of describing particular embodiments only, and is not intended to limit the scope of the invention. Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention.

The present invention, in some embodiments thereof, relates to systems and methods for facilitating and/or assisting in oral or dental measurements, and more specifically to methods and devices for recording occlusal or other dimensions and/or assist with imprinting a patient dentition.

An aspect of some embodiments of the present invention relates to an apparatus for registering occlusal dimensions. Occlusal dimension recording is commonly used for designing and manufacturing accurate dental artifacts so that a chosen (e.g., normal or aesthetic) occlusion can be achieved. Occlusal dimensions, as suggested in the present invention, may include but not be limited to a vertical occlusion dimension, but also aesthetic measurements recording such as midline, lip line and smile line. In some embodiments of the invention, the apparatus comprises at least one registration base member which includes a support surface at a first end and at least one connector at a second end opposing the first end.

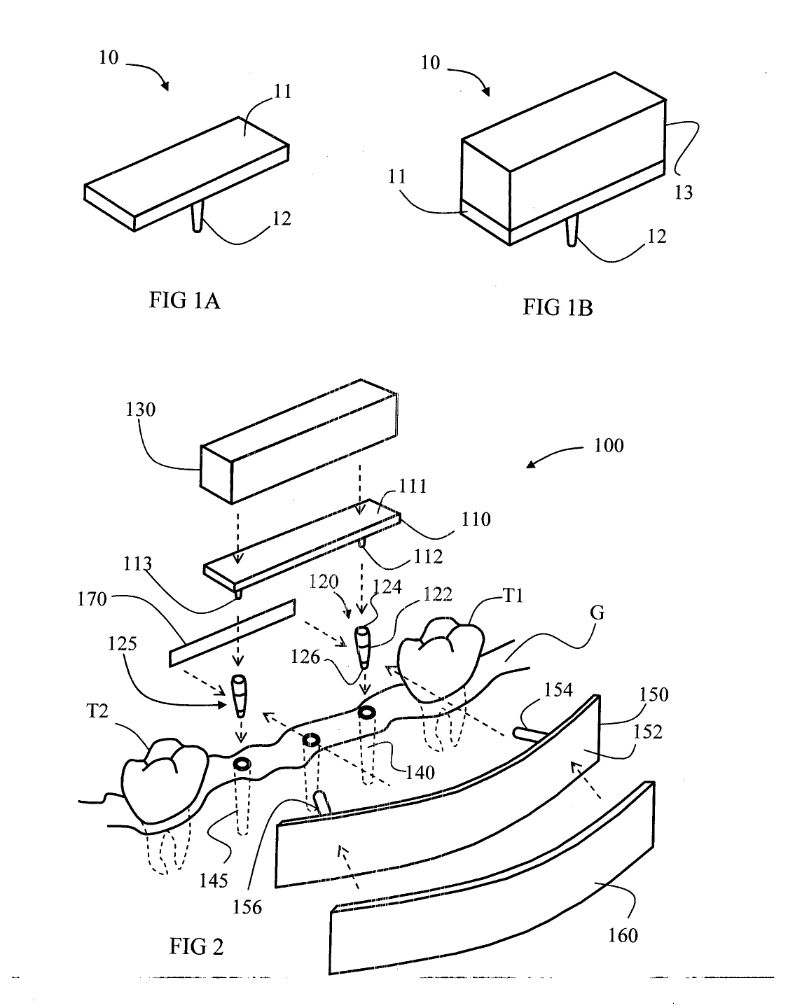

Now referring to the drawings, FIG. 1A illustrates a schematic isometric view of a registration base member 10, in an optionally general and/or simplified fashion, in accordance with embodiments of the invention, comprising a support surface 11 and a connector 12 outwardly projected from an opposing surface to support surface 12.

In some embodiments of the invention, registration base member 10 is designed such to be at least partially unyielding, resistive and/or rigid under compressive and other stresses arising from jaws closing thereupon. Alternatively or additionally, registration base member 10 is at least partly and/or partially deformable in at least one axis, in order to allow a user or practitioner to selectively fixate a chosen shape and/or curvature. Optionally, registration base member 10 may be bendable and/or malleable to a chosen contour. Optionally, registration base member 10 is elastically deformable or plastically deformable. Optionally, registration base member 10 is settable (e.g., curable) to fixate a chosen geometry. In some embodiments of the invention, registration base member 10 is shaped as a flat solid (i.e., being substantially thin with respect to length and/or width). Optionally, registration base member 10 comprises a mesh (not shown in FIG. 1A). In some embodiments of the invention, registration base member 10 includes an inscribable portion, optionally at support surface 11, thereby allowing a practitioner to mark and/or engrave markings (e.g., lines, dots and/or symbols) thereto. In some embodiments of the invention, registration base member 10 includes a cuttable or a breakable portion (e.g., a dot, a line, or an area) to thereby allow a practitioner to prepare a chosen or a predeterminedly allowed outline by removing portions thereof.

In some embodiments of the invention registration base member 10 readily facilitates and/or includes means (not shown) to not disrupt or to even improve imagery processes, such as CAD/CAM scanning or radiographic scanning (e.g., CT scan). In some embodiments, registration base member 10 includes a radiopaque portion and/or marker and/or identification marking. Optionally, registration base member 10 includes light absorbing and/or dissipating portion, optionally by including a color, a texture and/or a covering layer.

In some embodiments of the invention, apparatuses or registration base members of the present invention include at least one support surface which is sized and/or configured for providing areal support to a registration rim, in order to comply with forces and pressures of high magnitude projected towards the registration rim at occlusion and also to allow improved registering 2D or 3D coverage. Reference is now made to FIG. 1B which schematically illustrates an isometric view of registration base member 10 now deploying a registration rim 13 on support surface 11, in accordance with embodiments of the invention.

A registration rim, especially when provided for recording bite registrations, can be referred to as an occluding surface built on temporary or permanent denture bases for the purpose of making maxillomandibular relation records and for arranging teeth. Wax rim is commonly used in the registration stage in order to achieve three-dimensional prescription whereby the template of the intended denture is ‘prescribed and fashioned’ clinically before being dispatched to the laboratory for placement of the teeth on the trial denture.

Registration rim 13 may be a part or an areal projection originating from support surface 11, or it may be a distinct member detachable and/or connectable to registration base member 10 and at least partly supported with support surface 11. According to the present invention, registration rim 13 may be any material, design and/or fixture configured and applicable to record, copy and/or transfer, at least partially, imprints of bodily prominences, protrusions and/or curvatures, pressed thereto, including but not limited to an opposing tooth, a denture or other permanent or temporal dental artifact. Registration rim 13 may include a deformable imprinting block and/or an impression or registration material, including but not limited to wax, acrylic material, alginate, composite resins, silicones, Vinyl Polysiloxane (VPS), thermoplastic, polyether, and hydrocolloid. In some embodiments, the impression material readily fills a rigid container having an opening (shown in FIG. 5, respectively as container 2120 and opening 2140), which may or may not be provided with a removal covering. Registration rim 13 may include layers of impression material having a thickness, optionally a maximal thickness, of less than 10 mm, optionally less than 7 mm, or even optionally less than 5 mm.

Other types of registration rims, according to embodiments of the present invention may, alternatively or additionally, include other recording means (not shown) such as a plurality of sensors, optionally pressure microsensors, optionally piezoelectric (e.g., piezoresistive or piezocapacitive) sensors, optionally electrochemically reactive sensors, provided in a preferred coverage scheme on the support surface to thereby allow discrete and/or segmented areal measurement. In some embodiments of the invention, the apparatus includes means to receive, process, control and/or transmit the measured data and/or any information linked thereto to an outer system, for example a computer, a computerized scanner and/or a prototyping device; and the information may then be transferred wired and/or wirelessly.

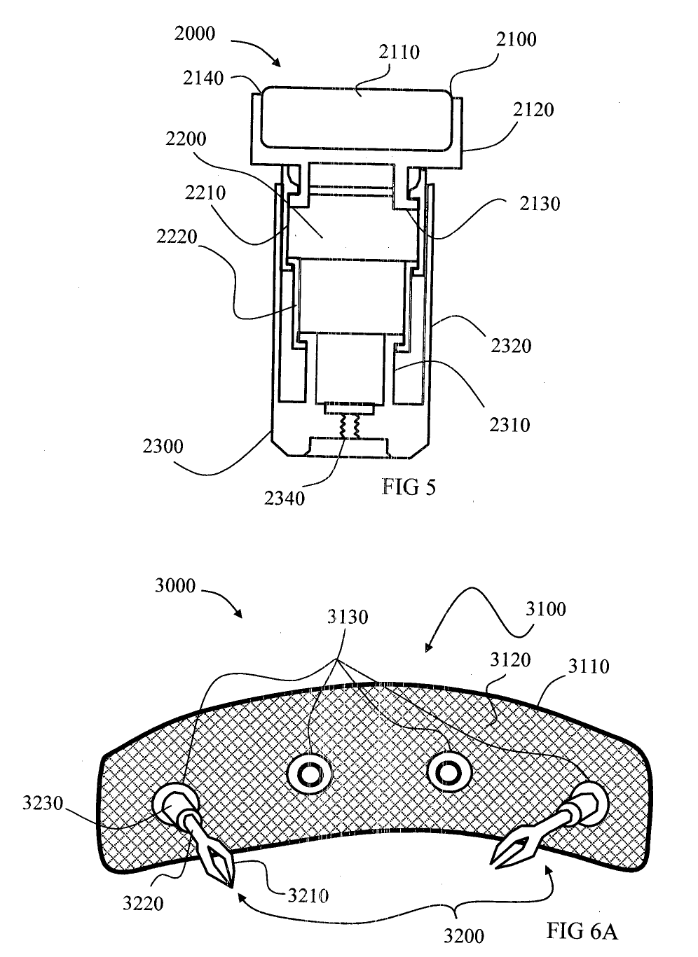

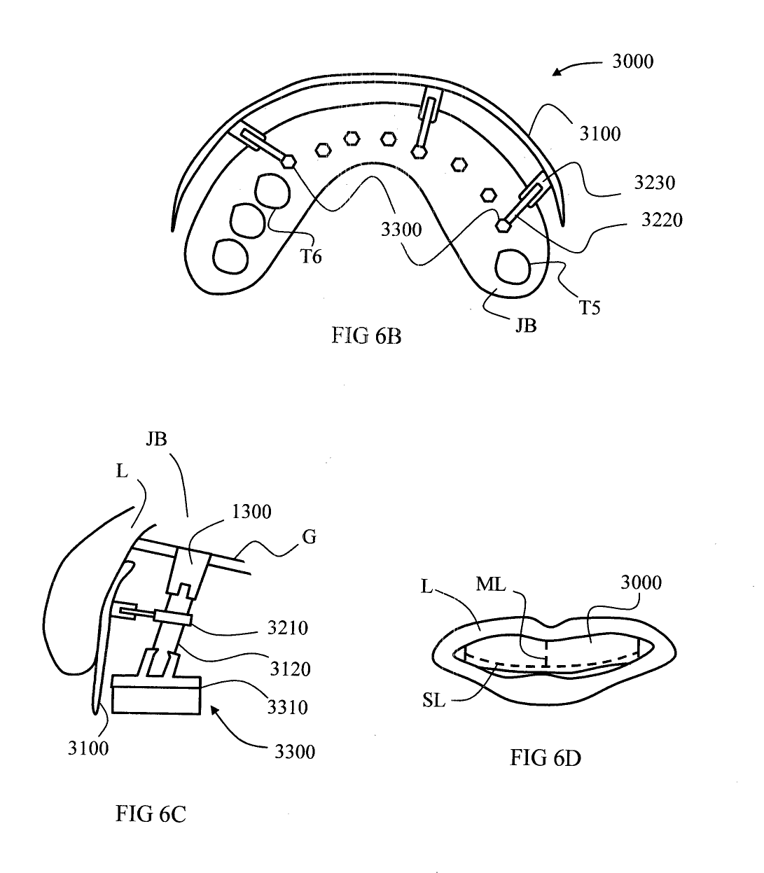

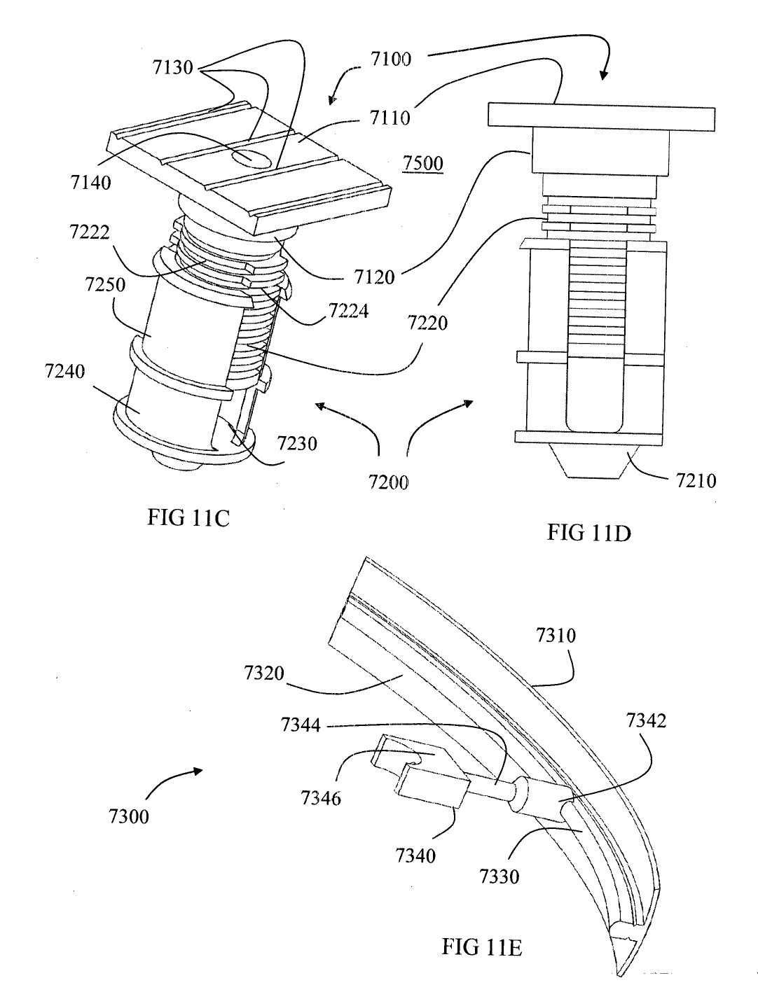

In some embodiments of the invention, registration base member 10 includes at least one connector 12 that is adapted to releasably connect to a dental implant extension (as, for example, shown in FIG. 2 and referred to as dental implant extension 120). Connector 12 may include any connection mechanism known to art, including but not limited to snap connection, quick release mechanism (e.g., skewer), quick connectors, bolting, and threading. In some embodiments, connector includes 12 ridges and/or recesses configured for slidably engaging a docking arrangement provided at a dental implant extension. Optionally, such a docking arrangement also facilitates proper positioning, aligning and/or locking/securing in-place. The docking arrangement may include at least one recess, protrusion, and/or ridge. Optionally, the docking arrangement is positionable (e.g., slidable) along a length of the dental implant extension. In some embodiments, connector 12 is movable (e.g., slidable) along a track provided is on a registration base member (such as 7330 in vestibular arch 7300, shown in FIG. 11). Alternatively or additionally, connector 12 is detachably connectable to different portions of the registration base member, either a track or any docking and/or locking arrangement (as for example snap fasters, as shown in FIG. 6A). In some embodiments of the invention, connector 12 is adjustable in length to thereby provide local distance between the interconnected registration base member and dental implant extension. In some embodiments, connector 12 reaches a predetermined length and/or is selectively adjustable by the user. In some embodiments, connector 12 includes a rod member slidably fixed to a housing lumen.

Reference is now made to FIG. 2 which illustrates a schematic exploded view of an exemplary modular system 100, in accordance with embodiments of the invention. In some embodiments of the invention, modular system 100 includes means for registering vertical occlusion dimensions (i.e., “bite registration”). Accordingly, modular system 100 includes a first registration base member hereinafter referred to as an occlusal flat solid 110, comprising an occlusal support surface 111 and two connectors 112 and 113 projected in an opposite direction to occlusal support surface 111.

In some embodiments of the invention, occlusal support surface 111 is sized and configured for covering an effective length and/or width of a portion of patient's gingiva G in-between laterally positioned tooth T1 and tooth T2, and/or for areally supporting a registration rim 130 over that gingival portion. In some embodiments, the distance between connectors 112 and 113 corresponds with a distance between bared openings of readily implanted dental implants 140 and 145, respectively. In some embodiments, Connectors 112 and 113 are distant one to the other, either fixedly or by setting a distance predeterminedly or selectively. Occlusal flat solid 110 may span over a length of optionally up to about 10 cm, optionally up to about 5 cm, optionally up to about 1 cm, or more, or less, or intermediate. Likewise, occlusal flat solid 110 may include a width covering the width of gingival G portion, optionally more or less its width, and may be, for example, between 3 mm to 2 cm. In some embodiments, occlusal flat solid 110 and/or registration rim 130 is sized for registering a single opposing tooth, or optionally a different chosen number of opposing teeth. In some embodiments of the invention, occlusal flat solid 110 is modularly extendable and/or deformably extensible, in length and/or width. In some embodiments, occlusal flat solid 110 effectively supports bite registration rim 130, optionally having a thickness less than 5 mm, optionally about 2 mm.

In-between any corresponding pair of connector-implant there is provided a dental implant extension, namely an extension 120 interconnectable between connector 112 and dental is implant 140, and an extension 125 interconnectable between connector 113 and dental implant 145. Connectors 112 and 113, once connected against free ends of corresponding extensions 120 and 125, are designed to provide occlusal support surface 111 in occlusal position.

For ease of description, exemplary embodiments provided hereinafter will refer specifically to extension 120, while considering extension 125 may be similar or different thereto. Dental implant extension 120 may be any projected point or surface elevated from gingiva G level and may be connected to the implanted dental implant 140 or originate from it. Extension 120 may be, for example, any of a transfer, an impression coping, an abutment, a guiding pin, and/or any projecting member occlusably connected to one of them. In some embodiments, extension 120 is length adjustable, optionally between about 1 mm to about 30 mm, optionally between about 3 mm to about 15 mm, optionally between about 5 mm to about 10 mm, or higher or lower or intermediate. In some embodiments, extension 120 includes a housing member having lumen and a moving member selectively positionable in that lumen and along its length (not shown in FIG. 2).

In some embodiments of the invention, extension 120 is angularly adjustable, optionally between 0° to 90°, optionally between about 0° to about 45°, optionally selectively changeable to discrete angles, including 0° and 20° (all angles with respect to the longitudinal axis of dental implant 140). In order that support surface 111 will optionally be able to remain in a chosen plane, for example an occlusal plane, means may be provided to registration base member 110 and/or to extensions 120 and/or 125, for leveling support surface 111, optionally selectively, predeterminedly, or automatically. In some embodiments of the invention, extension 120 includes locking means for fixating a chosen geometry, optionally following length and/or angular adjustment. In some embodiments of the invention, extension 120 includes means for specified assembly positioning and direction in-place, and for example includes a vestibular positioning side having orientation marking including but not limited to a flat surface, distinct coloring, and/or marking.

In some embodiments of the invention, a modular system according to the present invention is provided with means to provide occlusion and/or aesthetic related measurements of the vestibular region and/or surfaces. In some embodiments, modular system 100 includes a second registration base hereinafter referred to as a vestibular flat solid 150. Vestibular flat solid 150 includes a vestibular support surface 152 and a pair of connectors 154 and 156, outwardly projected away from vestibular support surface 152. Vestibular support 150 is laterally connectable to dental implant extensions 120 and 125, with corresponding connectors 154 and is 156, to thereby provide vestibular support surface 152 in vestibular position. In some embodiments, a vestibular registration rim 160, is connectable to vestibular support surface 152, and is configured for at least one of: imprinting, writing, engraving, etching, coining and cutting. In some embodiments, vestibular registration rim 160 optionally includes a thickness less than 5 mm, optionally about 1 mm. In some embodiments, vestibular flat solid 150 is configured to support a lip before, during and/or after a bite registration or any other occlusion related measurement recording. In some embodiments, vestibular support surface 152 and/or vestibular registration rim 160 is textured and configured to harmlessly press against supported soft tissue, for example labial mucosa and/or buccal mucosa. In some embodiments, vestibular flat solid 150 is formable to a chosen or predetermined shape or curvature.

In some embodiments, modular system 100 provides rapid connecting and/or releasing arrangements, for in-situ assembling a plurality of members into a patient-specific occlusal registering apparatus. Therefore, in some embodiments of the invention, modular system 100 includes at least one occlusal flat solid 110 provided with at least one connector, for example connector 112, for connecting to at least one dental implant extension, for example extension 120; and at least one vestibular flat solid 150 provided with at least two connectors 154 and 156 for connecting to at least two dental implant extensions, such as extensions 120 and 125. In some embodiments-of the invention, modular system 100 also includes a plurality of dental implant extensions, optionally provided in a dedicated kit or in a kit comprising occlusal flat solid(s) and/or vestibular flat solid(s) and/or connectors thereof.

In some exemplary embodiments of the invention, modular system 100 further comprising at least one fixating member connectable to at least one dental implant extension and adapted for fixating chosen tridimensional geometric relations between at least two dental implant extensions connected thereto. A fixating member 170, as shown in FIG. 2, is fixedly connectable to extensions 120 and 125. Once fixated, the solid arrangement of modular system 100, may be considered a patient-specific occlusal registering apparatus, and can later be withdrawn from parient's oral cavity and used for prototyping or for lab/technician follow-up work. Accordingly, fixating member 170 may be connectable to extensions 120 and 125 at or in-between two points along a length of each of the extensions, thereby resisting potential pivoting or twisting motion therebetween. In some embodiments of the invention, fixating member 170 is sized, shaped and/or configured for placement in lingual and/or palatal positioning. Alternatively or additionally, fixating member 170 is sized, shaped and/or configured for placement in occlusal and/or buccal positioning.

Modular system 100 may be applied, at least with some chosen members thereof, for performing occlusal dimension measurements, in accordance with an aspect of some embodiments of the invention. Following a registered healing period, the practicing medical practitioner (for example a general dentist, a prosthodontist, a surgeon, or any member of a relevant medical team) may uncover the proximal projections of the patient's dental implants, including implants 140 and 145. The healing time will mostly depend on the chosen treatment course, and may be between a few hours to a few months. Alternatively or additionally, the implants are immediately loaded. Healing caps (not shown) may be placed on top of the implants. Optionally, after approximately 10 days, the sutures can be removed, and another period of time is awaited until the soft tissue properly heals.

In some embodiment, the dentist measures and analyzes the proper height and orientation for each implant and chooses dental implant extensions having a chosen height, and optionally an angle. A plurality of dental implant extensions, in this example, extensions 120 and 125, may then be fastened to a corresponding number of dental implants, for example implants 140 and 145. The practitioner may then connect occlusal flat solid 110 over extensions 120 and 125, by which occlusal support surface 111 is positioned in an occlusion position away from implants 140 and 145. The practitioner can then make any necessary measurements according to a number of occlusions made by the patient, and that optionally adjust height of occlusal support surface 110 according to a chosen occlusion. In some embodiments, if an extension is so positioned for replacing an incisor tooth, then it is chosen as or being angulated with respect to the corresponding dental implant longitudinal axis, optionally at 20°. In the event of deploying a plurality of occlusal flat solids, each occlusal flat solid may be separately adjusted in height and/or angle. In some embodiments, adjustments may be accomplished by replacing at least one dental implant extension with another having a chosen length and/or angle, in case it is a non-changeable extension; or by changing its length and/or angle in case of adjustable extension. Once occlusal flat solid 110 is adjusted, and optionally locked in-position, the practitioner may then take final measurements and/or make further adjustments until reaching proper patient-specific vertical dimensions. In some embodiments, dental implant extensions 120 and 125 are fixated and interconnected with fixating member 170 in chosen tridimensional geometric relations. Optionally, fixating member 170 is placed in a lingual and/or palatal position. Alternatively, fixating member 170 is placed in occlusal and/or buccal position.

In order to perform the bite registration, the practitioner may then provide occlusal registration rim 130 on occlusal support surface 111 and ask the patient to close it jaws thereupon for imprinting opposing teeth and/or dental artifacts. In other instances, when single-tooth sized occlusal solid plates and/or registration rims are used, at least two, optionally at least three, occlusal flat solids and/or registration rims are needed for registrations in multi-teeth prosthodontic treatments. The practitioner may ask the patient to remain occlusion until hardening of a setting impression material.

In some embodiments of the invention, the medical practitioner laterally connects vestibular flat solid 150 to extensions 120 and 125. In some embodiments, connectors 154 and 156, which are provided with vestibular flat solid 150, are length adjustable, so that the practitioner can selectively modify local distance and/or shape vestibular flat solid 150 according to need. The practitioner may then take measurements and/or make further adjustments until reaching proper patient-specific occlusal and/or aesthetic dimensions (including, optionally, midline, lip position and/or smile line).

Exemplary Single-Tooth Bite Registering

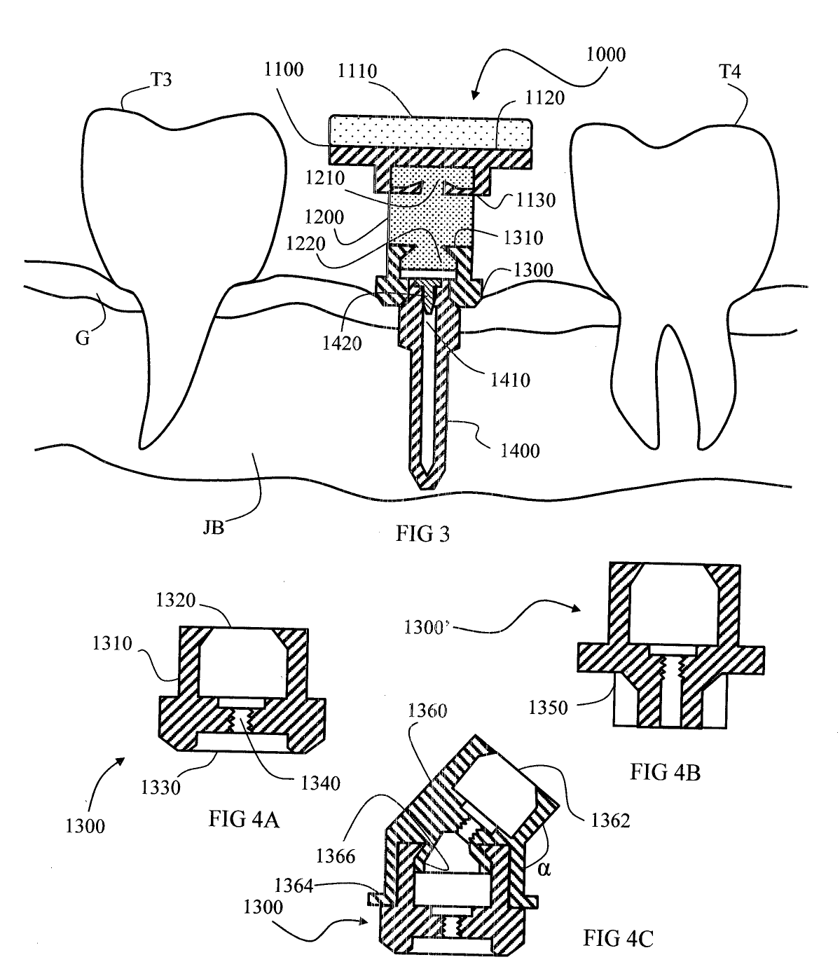

Reference is made to FIG. 3 which illustrates a schematic cutaway view of an exemplary occlusal registration device 1000, in accordance with embodiments of the invention. A practitioner may choose using a plurality of single-tooth occlusal registration devices, such as device 1000, optionally only 1 to 3 in each jaw, especially in multi-teeth prosthodontic treatments, thereby simplifying the procedure and allowing more flexibility for assembling in-vivo a modular system as previously described. Occlusal registration device 1000 shown in FIG. 3 is attached to a dental implant 1400 shown implanted in a jaw bone JB and surrounded and/or at least partially protruding out through gingiva G. Occlusal registration device 1000 includes an occlusal flat solid 1100 provided with an occlusal registration material 1110 (e.g., wax) placed on top of an occlusal support surface 1120. A transfer 1300 sits over and fixedly engaging the proximal protrusion of implant 1400 and affixed thereto with a bolt 1420 that is threaded in a threaded opening 1410 of implant 1400. A height coordinating element 1200 connects occlusal flat solid 1100 to transfer 1300. In an alternative design, only a single dental implant extension, such as a transfer or a height coordinating element, can be used to directly connect occlusal flat solid 1100 and implant 1400.

Occlusal flat solid 1100 is sized and proportioned to register an occlusion mark of a single opposing tooth (not shown). Wax or any other applicable material (for example, vinyl polysiloxane or acrylic resin) fills the tray and serves as registering rim 1110. In some embodiments, the material hardens over time and/or after contact with a different agent (e.g., water) so mark registration is permanently attained for laboratory purposes. Alternatively, occlusal flat solid 1100 includes pressure sensitive sensor(s) (not shown), disposed along occlusal support surface 1120, instead of registration rim 1110, which are designed for transmitting certain signals dependent on the amount of pressure, and its 2D/3D distribution, during occlusion.

In this particular exemplary embodiment, occlusal flat solid 1100 is set at an exact height using a particular height coordinating element 1200 having a fixed and/or predetermined size. As will be shown in later examples, height may be selectively set by using an alternative adjustable height mechanism. Inward bulges 1130 of occlusal flat solid 1100 are shown slidably affixed in corresponding recesses or slots 1210 in height coordinating element 1200. Similarly, inward bulges 1310 of transfer 1300 are shown slidably affixed in corresponding recesses or slots 1220 in height coordinating element 1200. Alternatively, occlusal flat solid 1100 and height coordinating element 1200 may be connected using snap-locking means or by any other known alternatives, including but not limited to threading and bolting. In some embodiments, the practitioner is provided with an instrumentation set comprising a number of height coordinating elements of different heights (and marked accordingly, e.g., by using different colors and/or annotations), from which he can choose a suitable one after taking measures and making analyses as appropriate. Alternatively or additionally, the practitioner can adjust height coordinating element 1200 by cutting it or assembling/disassembling parts thereof to a chosen height, when element 1200 may optionally include cutting or assembling line(s) along its length.

Transfer 1300 may be provided as an off-the-shelf part (e.g., a transfer, a coping or an abutment) or may have a special design for connectively fitting with a distal end of height coordinating element 1200. Transfer 1300 may include at its lower end non-rotational nesting means (e.g., hexagonal/pentagonal male-female coupling) and/or non-displacement means (e.g., bolting) to better fasten over implant 1400. An instrumentation set provided to the practitioner may include several designs of transfers, optionally differentiated by distal end coupling means and/or design to different forms of implant 1400. FIGS. 4A-B illustrate an enlarged view of female to transfer 1300 and an alternative male transfer 1300′. As shown in FIG. 2A, transfer 1300 includes a tubular body 1310 with a proximal portion encapsulating a lumen 1320, and a distal portion encapsulating a polygonal (e.g., hexagonal) recess 1330. In between lumen 1320 and recess 1330, there is provided a threaded through-hole 1340 intended for bolting transfer 1300 to implant 1400. Height coordinating element 1200 may be coupled with transfer 1300 by telescopically moving it into lumen 1320 and/or by nesting over the proximal portion of tubular body 1310, whereby a radially extended portion of body 1310 serves as its base. In FIG. 4B, transfer 1300′ is provided instead with a polygonal (e.g., hexagonal) distal portion 1350 which facilitates connection with a female type implant.

As previously described, there may be a need to connect occlusal registration device 1000 angled to the longitudinal axis of implant 1400. This is usually the case in upper incisor teeth which are naturally angled out of the jaw bone line. In some embodiments (not shown), height coordinating element 1200 is selectively bendable and/or curved and can be locked in a chosen angle or curvature. Alternatively, the practitioner is provided a kit containing height coordinating elements, differentiated by preset bending angles or curvatures. A third approach is shown in FIG. 4C, presenting an angular connector 1360 which may be applied for angularly connecting between straight height coordinating elements and transfers at a chosen or preset fixed angle. As shown the figure, angular connector 1360, angled to a, optionally approximately 20° (nonscalable in the figure), is secured to transfer 1300 at its lower end 1364 using snap connection 1366. A height coordinating element, such as 1200, may be telescopically positioned in an angled lumen 1362 and secured in-place similarly as in transfer 1300.

Exemplary Height Adjustable Occlusal Registering Device

In some instances, it may be preferable to allow selective height adjustment of the occlusal registration device in-vivo. This may be helpful, for example, in cases of multiple teeth replacement, where it is more complicated for the dentist and/or dental technician to assess an exact jaw/teeth closure for the developed crowns.

Reference is now made to FIG. 5 which schematically illustrates a cutaway view of an exemplary adjustable occlusal registration device 2000, in accordance with embodiments of the invention. In some embodiments, registration device 2000 includes a bite registering tray 2100 and an adjustable height mechanism 2200 readily combined with transfer base 2300. In some embodiments, height mechanism 2200 and base 2300 are detachably connectable one with the other or provided as an integral part. In some embodiments, registration tray 2100 is provided as a separate part and is connectable, optionally detachably, to a proximal portion of height mechanism 2200.

In some embodiments, bite registering tray 2100 includes a container or a frame 2120 which is provided filled with a registration material 2110 that allows coining or impressing of an opposing tooth or dental artifact prominence, via opening 2140, under occlusion forces. In some embodiments, frame 2120 is detachably connected to a proximal portion of height mechanism 2200, for example by snap-lock means 2130 which dismiss lateral and longitudinal displacement, and optionally further dismisses rotation movement. In an alternative design, frame 2120 and height mechanism 2200 are provided as a single member.

In some embodiments, adjustable height mechanism 2200 includes a plurality of concentric closed hollow members, for example proximal member 2210 and distal member 2220, that are telescopically slidable one over the other. Sliding may be substantially frictional or frictionless. Extension of height mechanism 2200, thereby elevation of member 2210 with respect to member 2220, can be set manually and selectively by the practitioner according to best practices to properly position bite registration tray 2100 with respect to an opposing tooth (not shown). Optionally, registration material 2110 is set in line with adjacent teeth height, or slightly elevated. In some embodiments, registration device 2000 may be locked in a chosen height while withstanding normal or abnormal occlusion forces. Locking mechanism (not shown) can be based on any mechanism known to art and may include, though not necessarily, rotational locking of member 2210 with respect to member 2220, a sliding bar lock (not shown), or other.

In some embodiments, height mechanism 2200 is at least partially enclosed in base 2300, and optionally (as illustrated) between inner wall 2310 and outer wall 2320. Walls 2310 and 2320 may be tubular and concentric one to the other, providing space in-between for members 2120 and/or 2220 sliding movements in and out. Additionally or alternatively, a spring (not shown) may be provided in the space between walls 2310 and 2320 that resists extraction and/or compression of height mechanism 2200. Optionally, the spring may be used as a shock absorber and/or a cushion. Alternatively or additionally, the space is filled with pressurized gas, thereby optionally designed to act as a pneumatic spring. In some embodiments, height mechanism 2200 is selectively settable between a first higher level (at free standing) and a second lower level (at maximal jaw closure or full occlusion). Transfer base 2300 may be designed and/or function similarly to as previously described transfer. In this particular figure, base 2300 is designed to accommodate a dental implant with a male proximal coupling. Bolting transfer 2300 to a dental implant is applicable via threaded lumen 2340 using a bolt (not shown) that can be securely threaded using an appropriate screw driver, that is insertable when bite registering tray 2100 is absent. After bolt securing, registering tray 2100 may be placed over and connected to height mechanism 2200.

Exemplary Vestibular Registration Device

Reference is now made to FIGS. 6A-D illustrating an exemplary vestibular flat solid 3000, and deployments thereof. In some embodiments, vestibular flat solid 3000 includes a body 3100 and a plurality of adjustable connectors 3200. In some embodiments, body 3100 includes a frame 3110 which hosts and secures a mesh or a net 3120. In some embodiments, frame 3110 is made of an elastic material that maintains a substantially rectangular or other polygonal shape. In some embodiments, net 3120 is substantially malleable and can take any given form along its surface. Optionally, net 3120 is elastic or super-elastic. Alternatively or additionally, net 3120 includes plastically deformable members which allow fixed relaxed formations of chosen shapes. In some embodiments, body 3100 further includes a plurality of coupling means (e.g., snap fasteners) 3130 for detachably connecting to the adjustable connectors 3200. In some embodiments, a registering material (e.g., wax) may be covering (not shown) at least one side of net 3120, optionally for registering anterior surfaces of adjacent teeth line and/or for protecting lip.

In some embodiments, each adjustable connector 3200 includes releasably connecting means 3210 for laterally connecting with a dental implant extension 3320 of an occlusal registering device 3300. Optionally, connecting means 3210 include grasping or snap-locking mechanism. In some embodiments, adjustable connector 3200 further includes at least two body members that can be selectively adjusted one with respect to the other thereby fixedly changing connecting means 3210 distance with respect to coupling means 3130. Optionally, and as shown in FIG. 6A, adjustable connector 3200 includes a telescopic length adjustable part comprising of a base member 3230 and a sliding extension member 3220. Locking means (not shown) may be used to lock each adjustable connector 3200 at a chosen extended length. Once attached to dental implant extension 3320, each adjustable connector 3200 can be selectively adjusted to a chosen length, while deforming overall body 3100 or only net 3120 to a chosen form.

Once properly attached in-place, vestibular flat solid 3000 may be line-marked and/or cut (e.g., by scissors or blade) according to need and facial, orthodontic and/or aesthetic proportions. As shown in FIG. 6D, a practitioner may draw a midline ML and smile lines SL that may cut through for properly adjusting shape of vestibular flat solid 3000 to the particular orthodontic surroundings.

Exemplary Method of Impression Recording

In aspect of some embodiments, there is also provided a method for imprinting a jaw, optionally but not necessarily using devices of the present invention and optionally following at least one of the steps described above.

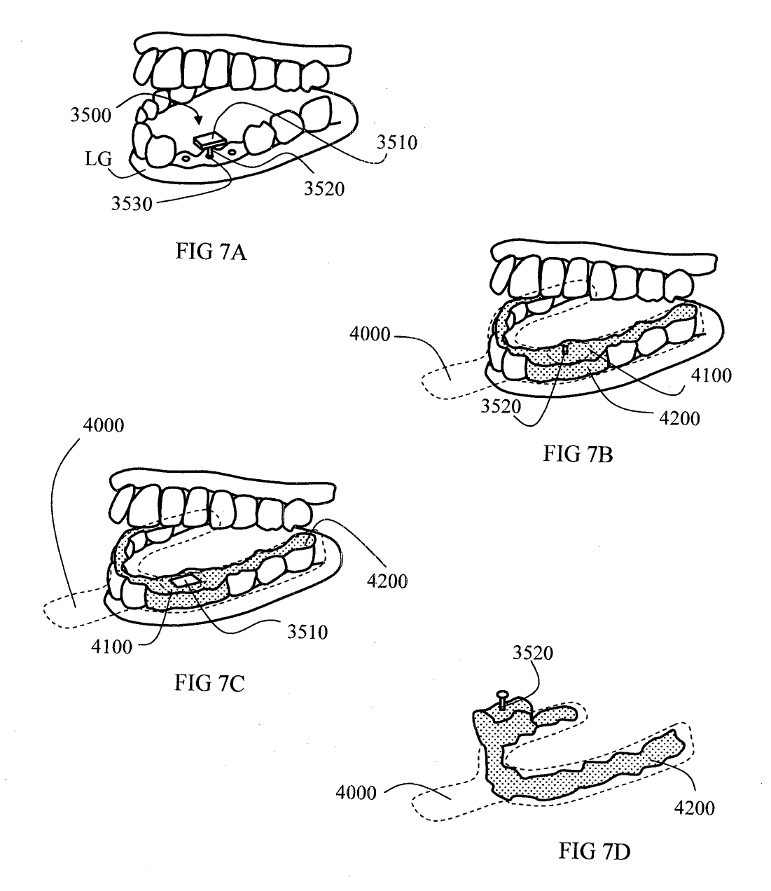

FIGS. 7A-D schematically illustrate several steps of dental impression using an exemplary occlusal registration device 3500 during multiple teeth replacement procedure, in accordance with embodiments of the invention. For exemplary purposes, the suggested scenario includes a lower jaw LG missing three adjacent native teeth and already installed with corresponding implants. In some embodiments, and as shown in FIG. 7A, registration device 3500 is provided connected to jaw LG, optionally following previous steps of measurements recording, as previously described. Registration device 3500 includes an occlusal flat solid 3510, connected to a dental implant extension 3520 interconnecting the occlusal flat solid 3510 to a dental implant 3530. Dental implant extension 3520 may be formerly adjusted and/or changed by chosen height and/or angle or may be used without considering height and/or angles.

In some embodiments, the practitioner presses, optionally moderately, an impression tray 4000, prefilled with impression material, against dental implant extension 3520. In a first scenario (shown in FIG. 7B) occlusal flat solid 3510 is initially removed and impression tray 4000 is pressed against the bare dental implant extension 3520. Alternatively (as shown in FIG. 7C), occlusal flat solid 3510 remains in place, with or without a registration rim connected thereto. In case that a registration rim is present, it may be covered before impression.

Any impression materials may be used, including but not limited to vinyl polysiloxane, polysiloxane, polyether, hydrocolloid, polysulfide, zinc oxide-eogenol paste or any combination thereof. Once the impression material sets (e.g., cured or hardens) to a final shape 4200, impression tray 4000 and final shaped impression material 4200 may then be withdrawn.

Impression tray 4000 may be previously adjusted to allow pressing against occlusal flat solid 3510 by cutting sized and/or shaped openings, such as opening 4100, at its base, which will correspond to and/or match over occlusal flat solid 3510 when pressed above it. If removed, occlusal flat solid 3510 may be kept in custody for further work, may be cleaned and prepared for future treatments, or may be discarded as disposable.

In some embodiments of the invention, dental implant extensions of the present invention may be used or include a coping, a transfer and/or a guide pin for the purpose of the impression taking. The suggested method may be utilized in an open tray technique, in which dental implant extension 3520 is unfastened but still connected to the corresponding dental implant 3530 before impression recording, so that it may be picked-up with the set impression material 4200 (as is shown in FIG. 7D). Alternatively, the suggested method may be utilized in a closed tray technique, in which dental implant extension 3520 may be detached and reattached to the set impression material 4200 after it had been removed.

In an alternative scenario, the practitioner may apply a different approach for impression taking without the use of an impression tray. According to another exemplary embodiment of the present invention, a method for imprinting a jaw includes: pressurizing settable impression material to covering a plurality of pre-deployed dental implant extensions, along a chosen bare gingival length; allowing the settable impression material to set (e.g., cure and/or harden); and removing the set impression material.

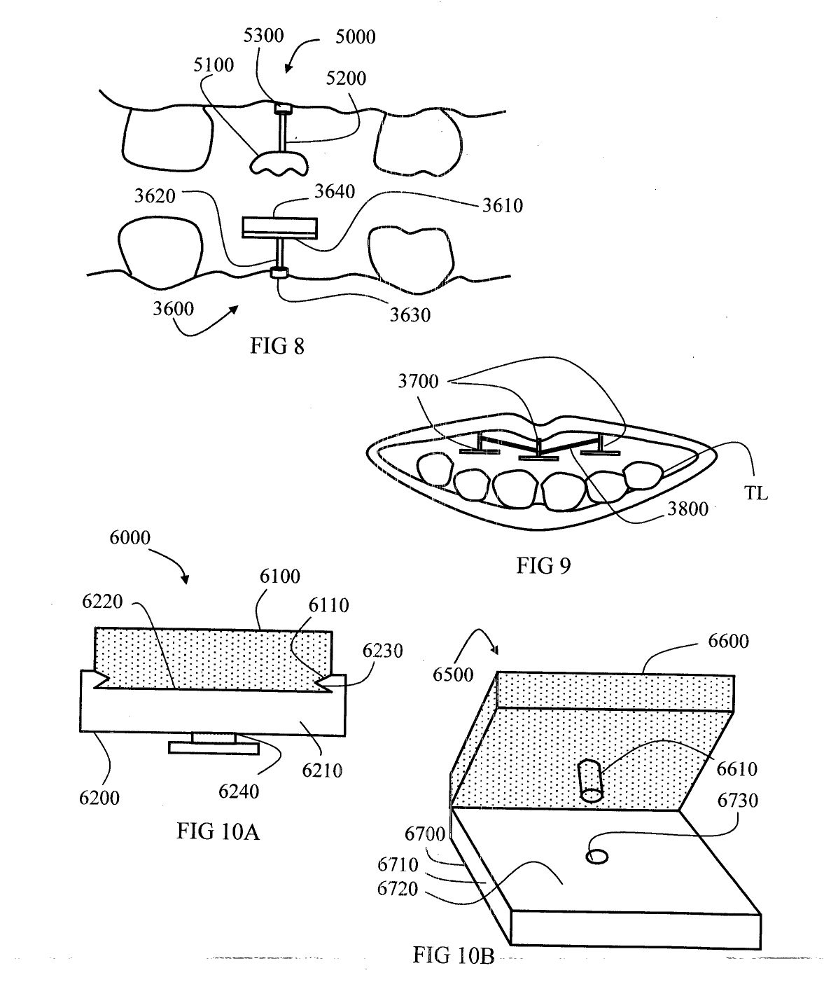

Reference is now made to FIG. 8 which schematically illustrates front view of an exemplary occlusal stamping device 5000 opposing an exemplary registering device 3600, deployed for imprinting two missing opposing teeth, in accordance with embodiments of the invention. Registering device 3600 is shown readily connected to a lower dental implant 3630 whereas stamping device 5000 is shown readily connected to a dental implant 5300, substantially opposing dental implant 3630. Registering device 3600 supports a registration rim 3640 with an occlusal flat solid 3610, as well as a dental implant extension that interconnects between flat solid 3610 and dental implant 3630. Stamping device 5000 is shown having a stamping head 5100 provided connected to dental implant 5300 via a connection rod 5200. In some embodiments, at least one of dental implant extension 3620 and connection rod 5200 is length adjustable. In some embodiments, heights of occlusal flat solid 3610 and/or of a stamping head 5100 are first adjusted as previously described. Then, the patient is requested to close his jaws thereby pressing stamping head 5100 onto occlusal flat solid 3610 until registration material hardens.

Reference is now made to FIG. 9 schematically illustrates exemplary interconnected occlusal registering devices 3700 deployed for impression an entire teeth line TL, in accordance with embodiments of the invention. In some embodiments, at least three registering devices, such as devices 3700, are needed for proper positioning in multi-teeth restorations. Each of the registering devices 3700 is selectively and independently set to a desired height in any of the alternatives previously described. In some embodiments, a fixating member 3800 is applied to connect at least one pair of registering devices 3700, thereby firmly positioning them in relation one to the other and in respect to oppose jaw. Once the registering devices 3700 are properly deployed, the patient may be requested to close his jaws thereby pressing devices 3700 towards TL for a proper 3-point bite registration. Occlusal registering devices 3700 and fixating member 3800 may be detached and removed from patient's mouth as a unitary fixated assembly for a is follow-up technician work.

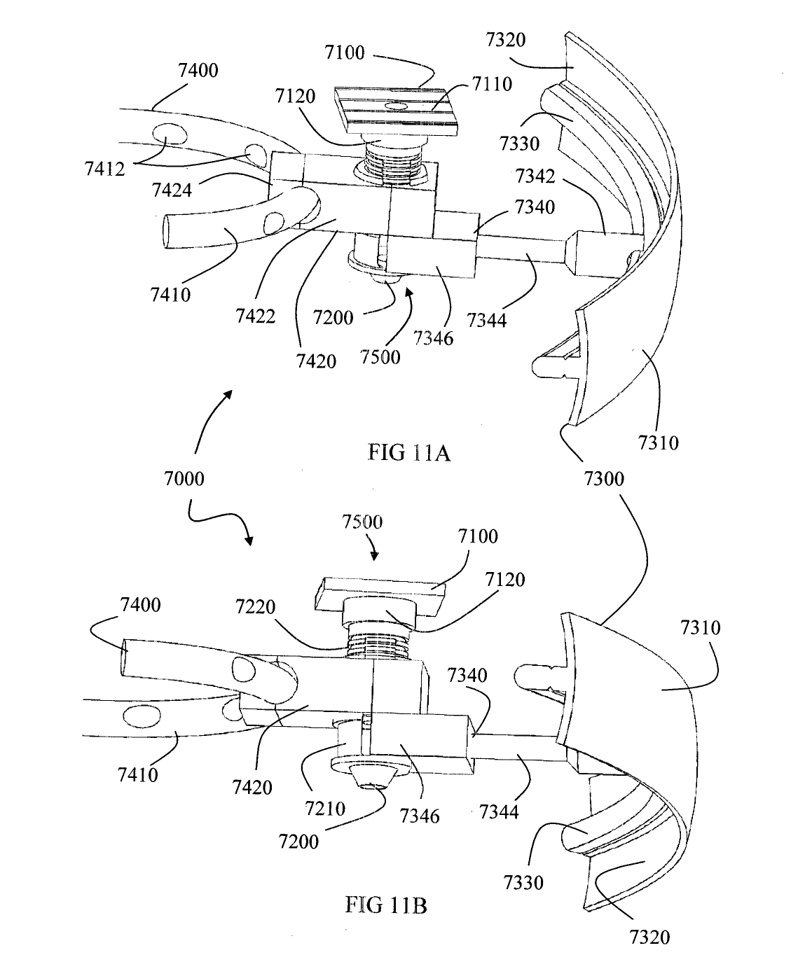

Reference is now made to FIGS. 10A-B which schematically illustrate views of exemplary connections elements, each connecting between a registration base member and an impression rim, in accordance with embodiments of the invention. FIG. 10A shows a registration device 6000 including a registration base member 6200 fixedly connected to an impression rim 6100. Registration base member includes a flat solid body 6210 having a support surface 6220 at an upper head and a slide-and-lock connector 6240 located at its opposing lower end. Two lateral inward protrusions 6230 are positioned slightly above support surface 6220. Impression rim 6100 includes two slots 6110 which corresponds with protrusions 6230, thereby slidably connectable to them. Such an arrangement may be preferred to avoid undesired pivoting and upward shifting of impression rim 6100. FIG. 10B shows a registration device 6500 including a registration base member 6700 fixedly connectable to an impression rim 6600. Registration base 6700 includes a body 6710 having a support surface 6720. Impression rim includes a downward protrusion 6610 that is slightly oversized to a lumen 6730 of body 6710. Pressing protrusion 6610 into lumen 6730 will therefore fixate registration base member 6700 and impression rim 6600, Such an arrangement may be preferred to avoid undesired sliding of impression rim 6600.

Exemplary Modular System

Reference is now made to FIGS. 11A-E which illustrate different views of an exemplary modular system 7000 incorporating at least one single-tooth occlusal registration device 7500, and members thereof, in accordance with embodiments of the invention. Besides occlusal registration device 7500, modular system 7000 further includes a vestibular arch unit 7300 and fixating member 7400. Occlusal registering device 7500 includes an occlusal flat solid 7100 and a length adjustable transfer 7200. FIGS. 11A-B show an assembly based on a single occlusal registration device 7500. FIGS. 11C and 11D show an isometric view and a side view of occlusal registration device 7500 and FIG. 11E shows an isometric view of vestibular arch unit 7300. Modular system 7000 is not shown with registration rims, occlusal or vestibular, although it can be provided with such, as previously described.

Occlusal flat solid 7100 comprises an occlusal support surface 7110 which is rectangularly shaped and sized to receive and areally support a corresponding rectangular registration rim, sized and configured for registering a single opposing tooth. An occlusal base connector 7120 projects outwardly away from occlusal support surface 7110 and is releasably connected (e.g., by snap locking or threading), to transfer 7200. Occlusal flat solid 7100 also includes elongated recesses 7130 and an opening 7140 provided on occlusal support surface 7110. Elongated recesses 7130 are designed to increase areal friction and bonding to an occlusal impression rim provided to occlusal support surface 7110. Opening 7140 allows at least one of: (a) delivering, fastening and/or unfastening a bolt attachable between transfer 7200 and a dental implant (similarly to as shown in FIG. 3); and (b) holding therein a possible outward protrusion of an occlusal registration rim (similarly to as shown in FIG. 10B). Occlusal flat solid 7100 may be comprised from a relatively rigid material such as stainless steel or plastic, and may be manufactured by machining, casting, injection molding or any other processing method.

Transfer 7200 is length adjustable and includes a hollow housing 7210 sized and shaped to nest over and detachably connect with a dental implant (not shown) and is adapted to host a toothed member 7220. Hollow housing 7210 and toothed member 7220 are selectively engagable to have an overall height that thereby determines height of occlusal support surface 7110 with respect to dental implant. Toothed member 7220 includes a plurality of parallel circumferential teeth, having a shallow portion 7222 and a protruding portion 7224 (as best shown in FIG. 11C). Toothed member 7220 is adapted to freely slide in housing 7210 when teeth protruding portion 7224 travel through a slot 7230 in housing 7210. At a certain chosen height, the practitioner can rotate toothed member 7220 clockwise or counterclockwise so that at least one protruded tooth portion 7224 engages in a corresponding lateral slot (not shown) provided in housing 7210 and locks in, optionally via snap locking. Transfer 7200 further includes grooved portions 7240 and 7250 for receiving slider-type vestibular connector 7340 and fixating member connector 7420, respectively.

Vestibular arch unit 7300 is an elastic normally curved arch shaped member that includes a vestibular support surface 7310 and an opposing surface 7320 upon which there is provided a track 7330 extended along its length. Track 7330 is an added part or a protrusion of vestibular arch unit 7300 and includes a substantially homogenous cross section with at least one slot provided along its length. A vestibular connector 7340 is transversely slidable along track 7330 by to engaging into the at least one slot. Vestibular connector includes a housing part 7342 and a moving part 7344 that is slidable within housing part 7342 and can be locked at any level. A snap grasper 7346 is provided at a distal end of moving part 7344 and is adapted to fixedly and releasably engage distal grooved portion 7240 of transfer 7200. Track 7330 is configured for attaching with a plurality of vestibular connectors such as connector 7340, whereas at least two vestibular connectors are needed to fixate vestibular arch unit 7300 in a patient specific position and curvature. By manipulating a length of each vestibular connector, such as connector 7340, vestibular arch unit can take different curvatures or wavy geometries, as needed.

Fixating member 7400 is an elastic, rigid or semi-rigid curved implement that includes a curved body 7410 having a plurality of ports 7412 evenly distributed along its length. Fixating member 7400 is provided with at least one fixating member connector 7420 that is assembled with a male member 7422 (releasably attachable to proximal grooved portion 7250) and a female member 7424 snap connecting therebetween over a chosen port 7412.

A practitioner may attach at least one occlusal registering device 7500 to an exposed head of a dental implant; then adjust height of each occlusal registering device 7500 by manually setting length of transfer 7200 as described above, until reaching requested patient's occlusion. All occlusal registering devices may then be affixed in relative position by connecting fixating member 7400 to them all. Occlusion registering rims may be then provided on each occlusal support surface 7110 recording opposing tooth/artifact imprint. A vestibular arch unit 7300 may be connected to at least two, optionally at least three of the occlusal registering devices 7500 and formed it to a chosen curvature by adjusting lengths of each vestibular connector 7340. The practitioner may record aesthetic lines and other measurements directly on vestibular support surface 7110 or to a vestibular registration rim (not shown) connected thereto.

In order to perform dental impression, the practitioner may disassemble and remove vestibular arch unit 7300 and optionally also any occlusal flat solid 7100. The practitioner may then pour or inject flowable setting material around each transfer 7200 and press thereupon an impression tray filled with impression material until setting thereof. Once impression tray is removed all transfers 7200 interconnected with fixating member 7400 are then removed and optionally shipped to technician laboratory (together with vestibular arch unit 7300, registration rims and any other necessary member) to continue work on the prostheses.

General

Unless otherwise mentioned, each device and/or component may be manufactured from any biocompatible material according to need. These materials may include, but are not limited to: stainless steel, aluminum, titanium or other biocompatible alloys; rubber, polyurethane, PEEK, or biocompatible polymeric materials.

All parts may be machined ad/or molded. Plastic parts may be injection molded.

Net and rim elements may include fibers, yarns, cables or other pliable parts which may comprise materials such as nylon, metallic strings, textiles, synthetic and high-performance polyethylene fibers such as Kevlar™, Dyneema™ and Spectral™, or others.

Several dimensions are mentioned throughout the specification, including:

“occlusal”—in the direction of the masticatory surfaces of the teeth;

“vestibular”—in the direction of outer surfaces of the teeth (including buccal and labial);

“buccal”—in the direction towards the cheeks away from tooth outer surface;

“labial”—in the direction towards lips away from tooth outer surface;

“lingual”—in the direction of the tongue away from tooth inner surface (lower teeth);

“palatal”—in the direction of the tongue away from tooth inner surface (upper teeth);

“proximal”—in the direction away from the bone jaw and towards practitioner hand;

“distal”—in the direction towards the bone jaw and away from practitioner hand.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An apparatus for registering occlusal dimensions, comprising:
a support surface sized and configured for providing areal support to a registration rim; and
at least one connector adapted to releasably connect to a dental implant extension.

2. An apparatus according to claim 1 comprising a registration base member having said support surface at a first end and said at least one connector at a second end opposing said first end.

3. An apparatus according to claim 2, wherein said registration base comprising an occlusal flat solid connectable against a free end of said dental implant extension thereby providing said support surface in occlusal position.

4. An apparatus according to claim 3, wherein said occlusal flat solid is sized and configured for positioning over a chosen bare gingival length.

5. An apparatus according to claim 4, wherein said occlusal flat solid is sized and configured for positioning in-between two laterally positioned teeth.

6. An apparatus according to 3, wherein said occlusal flat solid and/or said registration rim is sized for registering a single opposing tooth.

7. An apparatus according to claim 2, wherein said registration base member comprising a vestibular flat solid laterally connectable to said dental implant extension thereby providing said support surface in vestibular position.

8. An apparatus according to claim 7, wherein said vestibular flat solid is textured and configured to harmlessly press against supported soft tissue.

9. An apparatus according to claim 8, wherein said tissue comprising labial mucosa and/or buccal mucosa.

10. An apparatus according to claim 2, wherein said registration base member comprising a mesh.

11. An apparatus according to claim 2, wherein said registration base member comprising inscribable and/or cuttable portion.

12. An apparatus according to claim 1, wherein said registration rim comprising impression material.

13. An apparatus according to claim 12, wherein said impression material contains at least one of wax, acrylic material, composite resin, alginate, silicone, polyether, and hydrocolloid.

14. An apparatus according to claim 1, wherein said registration rim comprising a deformable imprinting block.

15. An apparatus according to claim 1, wherein said registration rim includes at least one pressure sensor.

16. An apparatus according to claim 2, wherein said registration base member and/or registration rim comprising radiopaque portion and/or marker.

17. An apparatus according to claim 2, wherein said registration base member and/or registration rim comprising identification marking.

18. An apparatus according to claim 2, wherein said registration base member and/or registration rim comprising light absorbing and/or dissipating portion.

19. An apparatus according to claim 12, wherein said impression material readily fills a rigid container having an opening.

20. An apparatus according to claim 19, wherein said opening is provided covered with a removable covering.

21-49. (canceled)
